(19) United States
(12) Patent Application Publication
(10) Pub. No.: US 2011/0190810 A1
(43) Pub. Date: Aug. 4, 2011

(54) MEDICAL GRIPPER INSTRUMENT

(75) Inventor: Andreas DINGLER, Birkenfeld (DE)

(73) Assignee: RICHARD WOLF GMBH, Knittlingen (DE)

(21) Appl. No.: 13/014,889

(22) Filed: Jan. 27, 2011

(30) Foreign Application Priority Data
Jan. 29, 2010 (DE) 10 2010 006 201.4

(51) Int. Cl. A61B 17/28

(52) U.S. Cl. 606/205

(57) ABSTRACT

A medical gripper instrument with two jaw parts which include distal gripping sections lying opposite one another. At least one of the jaw parts includes a proximal holding section which is elastically deformable in a manner such that the distal gripping sections of both jaw parts are capable of being moved to one another and away from one another by way of elastic deformation of the at least one holding section.

### BACKGROUND OF THE INVENTION

The present invention relates generally to a medical gripper instrument according to the preamble of the independent claim(s). Medical gripper instruments, in particular endoscopic gripper instruments, are known for different purposes and comprise moving jaw parts for gripping objects or tissue parts with operations. For example, medical gripper instruments in the form of needle take-over instruments are known, which are applied with endoscopic instruments for suturing work, in order to grip needles and to guide them for the suturing of wounds. A block is mostly provided with the known needle take-over instruments, which must be activated or deactivated with each gripping.

This renders the handling awkward. Moreover, such instruments are complicated with regard their construction and are thus expensive.

It is an objective of the present invention to provide an improved medical gripper instrument, in particular an endoscopic medical instrument such as a needle take-over instrument, which permits a secure holding for example of a thread or needle, without having to actuate a block. Moreover, the instrument of the present invention is to be as simple as possible with regard to its construction and inexpensive to manufacture.

### BRIEF SUMMARY OF THE INVENTION

The above objective is achieved by a medical gripper instrument with the features specified in the independent claim(s). Preferred embodiments are to be deduced from the dependent claims, the subsequent description as well as the attached drawings.

The medical instrument according to a preferred embodiment of the present invention in particular is an endoscopic medical instrument which is applied for endoscopic operations for gripping and handling tasks in the field of the operation. Particularly preferably, with regard to the medical gripper instrument, it is the case of a take-over instrument, such as a needle take-over instrument which is used for gripping a needle or a thread on suturing.

The gripper instrument according to a preferred embodiment of the present invention at its distal end comprises a jaw which is formed from two jaw parts which in each case have a gripping section. Thereby, in each case the distal section of the jaw part forms the gripping section. The two gripping sections lie opposite one another, so that one may grip things such as a needle and thread or tissue sections, between the two gripping sections. According to a preferred embodiment of the present invention, at least one of the jaw parts, apart from the gripping section, comprises a holding section situated on the proximal side of the gripping section. Thereby, the gripping section and the associated holding section of the jaw part are preferably connected in a fixed manner and in particular the gripping section and the holding section of the jaw part are preferably designed as one part or as one piece. This permits an inexpensive manufacture and assembly, since the number of necessary individual parts is kept small.

The holding section is designed in an elastically deformable manner. Thereby, the holding section at least of one of the jaw parts is elastically deformable in a manner such that a deflection of the associated gripping section connected to this holding section, is possible due to the elastic deformation. The elastically deformable or elastically bendable holding section of the jaw part thus assumes a joint function, so that it is possible to design the jaw of the gripper instrument in a joint-free manner. Inasmuch as this is concerned, it is possible to move the two gripping sections relative to one another and/or relative away from one another, alone on account of the elastic deformation of the at least one holding section. For example, depending on the design, the jaw which is formed between the gripping sections, may be opened and/or closed by way of the elastic deformation at least of a holding section. The joint-less design simplifies the construction of the instrument since in turn, the number of necessary individual parts and assembly steps may be reduced by way of this. Moreover, such an instrument is simpler to clean and to disinfect on account of the reduced number of individual parts and thus the reduced number of gaps between the individual parts.

Depending on the design, the jaw may be designed such that it is opened in the idle position and is closed by way of the deformation of the at least one holding section. Alternatively, it is also possible to design the jaw such that the gripping sections of the two jaw parts bear on one another in the idle position and are movable apart by way of the elastic deformation of at least one holding section. The jaw between the gripping sections is opened due to this deformation of the holding section. This is the preferred design for a needle take-over instrument. A needle which has once been received or a thread which has once been received, are thus led between the two gripping sections in the idle position of the instrument, due to the fact that the instrument is closed in its idle position.

According to a preferred embodiment of the present invention, both jaw parts, apart from a gripping section, in each case comprise a proximal holding section which is elastically deformable in a manner such that the gripping sections of the two jaw parts are movable to one another and/or away from one another by way of the elastic deformation of the two holding sections. Here too, it is preferable for the gripping section of a jaw part and the holding section which is adjacent on the proximal side, to be connected to one another in a fixed manner, preferably as one piece or one part, in order to reduce the number of required individual parts. Preferably, the two jaw parts are designed symmetrically to one another with respect to the instrument longitudinal axis, so that they are moved symmetrically apart or to one another for opening and closing the jaw. With this embodiment too, the jaw parts may be designed such that they are closed in their idle position and are moved apart by way of deformation of the holding sections. A design the other way round is also conceivable, with which the jaw is opened in its idle position and is closed by way of deformation of the holding sections.

Due to the elastic deformability of the holding sections, one succeeds in these being able to be deformed by way of the action of an external force, in order to open or close the jaw, depending on the design of the jaw. As soon as this external force is released, the holding sections and thus the gripping sections connected to them, move back into their initial or idle position due to the elasticity of the material of the holding sections. Thus, the elastic deformation ability of the holding section not only assumes the joint function, but at the same time forms a restoring spring.

Particularly preferably, at least one holding section of a jaw part is already elastically deformed in the idle position, in a manner such that it produces a spring force which holds the gripping sections of the two jaw parts in bearing contact. Preferably, also with this design, both jaw parts comprise a corresponding elastically deformable holding section and both holding sections are elastically deformed in their idle position, so that they are biased to one another and hold the two joint parts in bearing contact amid biasing. In this manner, a holding force or clamping force may be produced between the two gripping sections in the idle position, for example in order to be able to securely hold a thread and a needle, in the idle position. Thus, one may make do without complicated blocking mechanisms. As a whole, the jaw parts and their holding sections are thus designed in the manner of a leaf spring. Thereby, the jaw parts are designed such that in their initial condition, i.e. when they are not assembled, they are shaped or curved such that the gripping section of the jaw part extends beyond the later bearing contact plane with the second jaw part, so that it is situated at the opposite side of this bearing contact plane compared to its associated holding section. If one or both jaw parts are designed accordingly, so that their gripping sections are aligned to one another, this has the effect that when the two jaw parts are assembled in the instrument, the two gripping sections thus come into bearing contact such that the holding sections are elastically deformed by a certain amount, so that the two gripping sections in each case come into bearing contact on the same side of the bearing contact plane as their associated holding section, and the bearing contact plane of the two gripping sections is situated preferably exactly on the instrument axis. In this manner, a biasing is produced in the holding sections, which effects a holding force between the two gripping sections. For opening the forceps jaw, the holding sections must be further deformed, so that the gripping sections in each case are moved away from the middle plane or the bearing contact plane. It is to be understood that accordingly, one may also form an angled jaw. With this, the bearing contact plane then extends angled to the instrument longitudinal axis.

For opening the jaw, preferably a wedge-like actuation element is present, which is movable linearly in the direction of its tip, between the jaw parts, in a manner such that the gripping sections of the two jaw parts are moved apart amid the elastic bending of at least one holding section of a jaw part. With this design, thus the gripping sections of the jaw parts in their idle position are in bearing contact, preferably in a biased bearing contact, with which at least one holding section produces a spring force, by way of which the two gripping sections are pressed on one another. The wedge-like actuation element is this movable between the jaw parts, in particular their holding sections, such that proceeding from its tip, with a further linear movement, on account of its wedge shape, it presses the two jaw part and thus their gripping sections apart, so that the jaw formed between the gripping sections is opened.

In order to produce this movement, at least one of the jaw parts which is deformable or deflectable, comprises an actuation surface with which the actuation element may be brought into contact, for moving the gripping section of the jaw part. In the idle position, the distance of this actuation surface to the instrument longitudinal axis is smaller than the widest distance of the wedge surface to this axis. The oblique wedge surface of the actuation elements, with its moment in the axial direction, slides on this actuation surface. In order to keep the friction as low as possible, the actuation surface may be designed as small as possible. Ideally, the surface is so small, that only a point contact or a line contact is formed. For example, the actuation surface may be moved only by an edge in the transition region between the holding section and the gripping section of the deflectable jaw part. With this design, a recess may be formed in the holding section, in which recess the actuation element is completely received in its idle position. With a linear advance, it then comes to bear with its wedge surface on an end-edge of the recess which is situated in the transition region to the gripping section, wherein this edge forms the actuation surface. Preferably, this actuation surface is designed in a rounded manner.

If both jaw parts are accordingly deflectable, preferably such an actuation surface is provided on each of the jaw parts, wherein these lie opposite one another and, as the case may be, are inclined or bent oppositely to one another, in order to come into bearing contact with two wedge surfaces of the actuation element which are inclined opposite to one another.

For closing the jaw of the instrument, the actuation element is moved back again in the linear direction, wherein the jaw parts then automatically move back into their idle position due to the elastic deflection of the holding sections. This elastic deflection of the holding sections effects an elastic stress in the holding sections, which ensures that the holding sections tend to automatically return back into their initial position.

An actuation rod which is movable in the direction of the instrument longitudinal axis and which is connected at its proximal end to a handle, is provided for actuating the instrument jaw formed by the gripping sections, further preferably, for moving the gripping section of at least one of the jaw parts. The handle may be connected or coupled to the actuation rod in a direct or indirect manner, e.g. via a leaf spring element, as is described hereinafter. This actuation rod thus extends along the instrument longitudinal axis from the proximal end of the instrument, at which the handle is situated, to the distal end of the instrument, at which the instrument jaw is formed by the two gripping sections. By way of a linear displacement of the actuation rod, at least one, preferably both gripping sections are moved. For this, an actuation element according to the preceding description is preferably arranged at the distal end of the actuation rod, so that the actuation element is moved further between the two jaw parts by way of a linear displacement of the actuation rod in the distal direction, in order to move these jaw parts apart transversely to the movement direction of the actuation rod. For closing the jaw, the actuation rod and thus the actuation element connected to its distal end are moved back again in the proximal direction, wherein the jaw automatically closes by way of the movement of the jaw parts due to the elastic restoring forces in the holding sections.

According to a preferred embodiment of the present invention, the gripper instrument comprises a handle which has at least one gripping limb which at its distal end is mounted in an articulated manner, and at its proximal end is connected to a leaf spring element which, proceeding from the proximal end of the gripping limb, extends distally, and with its distal end distanced to the gripping limb, is connected to the proximal end of an actuation rod. The leaf spring element lies in the inside of the handle and is thereby attached, preferably in a fixed manner, to the proximal end of the gripping limb and extends from there in the distal direction in a manner such that it distances itself further away from the gripping limb, along its extension. At its distal end, the leaf spring element may be connected directly or indirectly to the proximal end of the actuation rod. Thereby, the leaf spring element is connected, preferably in an articulated manner, to the proximal end of the actuation rod. The leaf spring element assumes two functions. On the one hand, it transmits a movement of the gripping limb onto the actuation rod, so that on pivoting the gripping limb, the actuation rod is moved linearly, i.e. in the direction of the instrument longitudinal axis. Preferably, on pivoting the griping limb inwards, i.e. towards the instrument longitudinal axis, the spring element is moved and deformed such that the connection point with the actuation rod is displaced distally and thus also the actuation rod is moved in the distal direction. On the other hand, the spring element simultaneously assumes the function of a restoring spring. For moving the actuation rod, the leaf spring element is elastically bent or deformed on pivoting the gripping limb. Due to this elastic deformation of the leaf spring element, a restoring force is produced, which on releasing the pressure on the gripping limb, moves this back into its initial position, wherein simultaneously the actuation rod is also moved back into its initial position.

Preferably, the handle comprises two such gripping limbs in each case with such a leaf spring element. The leaf spring elements lie between the two gripping limbs in the inside of the handle. The two gripping limbs are mounted in an articulated manner at a common or at an in each case separate articulation point. Thereby, the articulation points preferably lie symmetrically to the instrument longitudinal axis, and in particular the complete handle is designed mirror-symmetrically to the instrument longitudinal axis. Accordingly, also two leaf spring elements are provided with this embodiment, which are connected preferably at a common articulation point, to the proximal end of the actuation rod, in an articulated manner. This articulation point thereby preferably lies on the symmetry line of the handle, i.e. preferably on the instrument longitudinal axis. The leaf spring elements are designed curved in their idle position, so that, proceeding from the proximal end which is situated distanced to the instrument longitudinal axis, they curve towards the instrument longitudinal axis and thereby distance themselves further from the inner side of the associated gripping limb. If the gripping limbs are move to one another, this leads to a bending of the leaf spring elements, so that these assume a stretched form, by which means the articulation point on the actuation rod is displaced distally.

Further preferably, the connection of the leaf spring element to the proximal end of the actuation rod is distanced to the articulated mounting of the gripping limb in the proximal direction. The gripping limbs and leaf spring elements thus pivot about different articulation points. Thereby, the leaf spring element in the direction of the instrument longitudinal axis, is designed shorter than the gripping limb, so that with a pressure on the gripping limb, the above-described bending or stretching of the leaf spring element or elements occurs, and the actuation rod is displaced axially.

### BRIEF DESCRIPTION OF THE SEVERAL VIEWS OF THE DRAWINGS

The foregoing summary, as well as the following detailed description of preferred embodiments of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown. In the drawings:

FIG. 1 is a side elevation view of an entire medical gripper instrument in the form of a needle take-over instrument according to a preferred embodiment of the present invention;

FIG. 2 is a magnified side elevation view of a distal end of the instrument according to FIG. 1, with an opened jaw;

FIG. 3 is a magnified detail view of the opened jaw according to FIGS. 1 and 2;

FIG. 4 is a partial longitudinal section view of the instrument according to FIG. 2;

FIG. 5 is a cross-section view along line V-V in FIG. 2; and

FIG. 6 is a cross section view along line VI-VI in FIG. 2.

### DETAILED DESCRIPTION OF THE INVENTION

Certain terminology is used in the following description for convenience only and is not limiting. The word “inwardly” refers to a direction toward the geometric center of the device, and designated parts thereof, in accordance with the present invention. Unless specifically set forth herein, the terms “a,” “an” and “the” are not limited to one element, but instead should be read as meaning “at least one.” The terminology includes the words noted above, derivatives thereof and words of similar import.

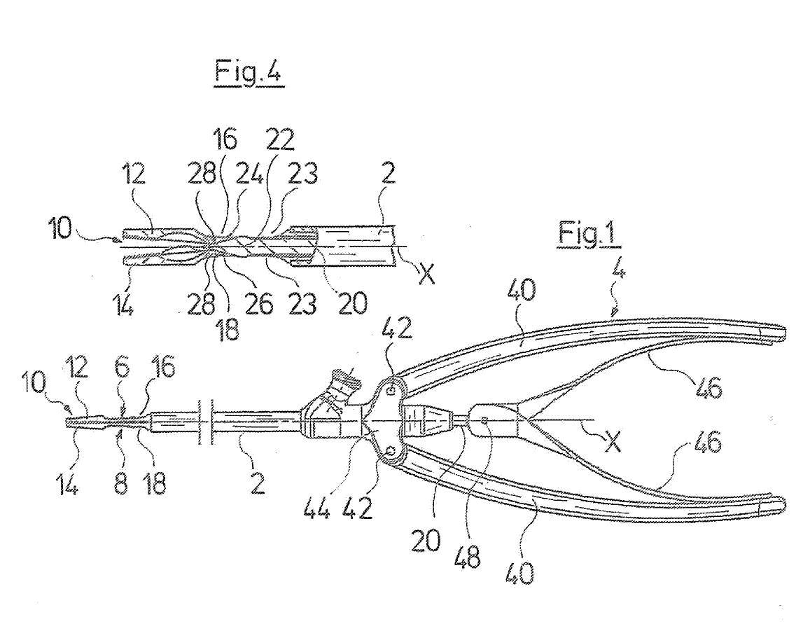

Referring to the drawings in detail, wherein like numerals indicate like elements throughout the several views, the medical gripper instrument shown in the figures is a needle take-over instrument. The instrument preferably comprises a shank 2, at whose proximal end a handle 4 and at whose distal end a jaw formed of two jaw parts 6 and 8 are situated. In the idle condition shown in FIG. 1, the jaw parts 6 and 8 bear on one another with their gripping sections 12 and 14, so that the jaw 10 is closed.

According to a preferred embodiment of the present invention, the jaw 10 is designed in a jointless manner with the shown instrument. For example, there is no joint, in which rigid jaw parts are pivotably articulated. Rather, here the jaw parts 6 and 8 are formed of a resilient or elastic material, preferably metal, and the deflection of the jaw parts or in particular their gripping sections 12 and 14 for opening the forceps jaw, is effected by way of elastic deformation of the jaw parts 6 and 8.

The jaw parts 6 and 8 at their distal end in each case comprise a gripping section 12, 14. On the proximal side, in each case a holding section 16, 18 connect to these gripping sections 12 and 14. The holding section 16 is designed as one piece with the gripping section 12, and the holding section 18 is designed as one piece with the gripping section 14. The holding sections 16 and 18 are in each case connected at their proximal end to the distal end of the shank 2 in a fixed manner.

In the idle position shown in FIG. 1, the two jaw parts 6 and 8 bear on one another with the inner surfaces facing one another and parallel to the instrument longitudinal axis X. In order, in this condition, to ensure a firm bearing contact of the gripping sections 12 and 14, the jaw parts 6, 8 may in each case be shaped, such that in their initial shape, in which they are not assembled on the shank 2, they are shaped in each case such that the holding sections 16, 18 and, as the case may be, the gripping sections 12, 14, are designed curved, so that the inner surface of the jaw part 6, 8 which is envisaged for bearing contact on the oppositely lying jaw part, essentially has a concave curvature in this initial position. By way of this, one ensures that on assembly of the two jaw parts 6 and 8 on one another, an elastic deformation of the holding sections 16, 18 is already necessary, in order to stretch the jaw parts and to bring them into the straight shape shown in FIG. 1. By way of this elastic deformation, a biasing force is produced in the holding sections 16, 18 of the jaw parts 6, 8, which holds the two gripping sections 12, 14 in bearing contact.

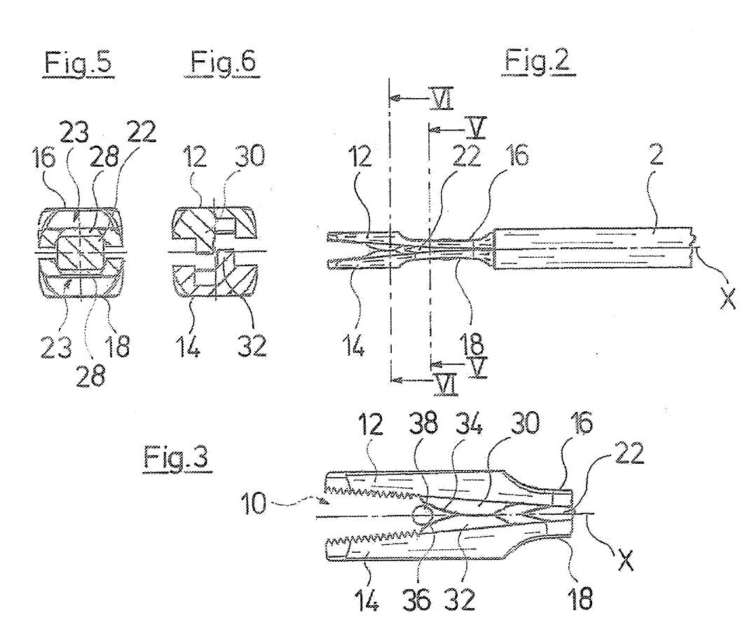

For opening the jaw 10, the instrument comprises an actuation rod in the form of a push rod 20. The push rod 20 at its distal end comprises an actuation element 22 which is designed in a wedge-like manner. The wedge-like actuation element 22 tapers to the distal end of the push rod 20 and in this manner has to wedge surfaces or pressing surfaces 24, 26 which are inclined to one another. The pressing surfaces 24, 26 are inclined such that they contact one another at a tip which lies on the instrument longitudinal axis X corresponding to the movement direction of the push rod 20. The pressing surfaces 24, 26, when the push rod 20 is advanced in the distal direction, come into contact with the inner surfaces 28 (see FIG. 5) of the jaw parts 6, 8 in the transition region between the holding sections 16, 18 and the gripping sections 12, 14. The wedge-like actuation element 22 is in each case situated in a recess 23 of the respective holding section 16, 18. The inner surfaces 28, which form the actuation surfaces or edges, represent the distal end-edges of this recess 23. These are rounded, so that they form as a narrow as possible bearing contact surface on the pressing surfaces 24, 26 with as little friction as possible. Thus, the inner surfaces or actuation surfaces 28 slide on the pressing surfaces 24, 26. In this manner, with a further advance of the push rod 20, the gripping sections 12, 14 are pressed apart in a direction transverse to the instrument longitudinal axis X, and the jaw 10 opens as is shown in the FIGS. 2, 3 and 4.

In the region of the gripping sections 12, 14, return elements 30, 32 are formed on the jaw parts 6,8, situated towards the proximal end of the gripping sections 12, 14. With regard to these, it is the case of inwardly, i.e. in the direction of the other jaw part, projecting projections. Thereby, the return elements 30, 32 are arranged such that they are offset in a direction transverse to the instrument longitudinal axis X, so that they may move past one another on closing the jaw 10. Thereby, the return elements 30, 32 however also overlap in the opened condition of the jaw. Ideally, the two jaw parts 6, 8 are designed in an identical manner and the return element 30 and 32 is laterally offset in each case out of the middle plane of the jaw part 6 and 8 respectively, so that with the assembly of two such jaw parts opposite one another, the described offset of the two return elements 30, 32 occurs and they may be moved past one another. At the distal end, the return elements 30, 32 comprise contact surfaces 34, 36 running obliquely to the instrument longitudinal axis X and which, on closing the jaw 10 when the gripping sections 12, 14 move to one another, serve to move a needle 38 which, for example, is situated between the jaw parts 6, 8, distally into the region of the gripping sections 12, 14, in which they may be held in a secure manner.

The jointless design of the jaw parts 6, 8 has the advantage that the number of individual parts is reduced, the cleaning is simplified and moreover a roughly constant holding force may be ensured over the complete opening range of the jaw 10.

The handle 4 as the jaw 10, in this example, is designed symmetrically to the instrument longitudinal axis X and comprises two gripping limbs 40 which in each case are mounted in an articulated manner at an articulation point 42 on a grip receiver 44 formed at the proximal end of the shank 2. Thereby, the joint points 42 for the two gripping limbs 40 are distanced in a direction transverse to the instrument longitudinal axis X. At their proximal end, the gripping limbs 40 are in each case connected to a leaf spring element 46. The connection between the gripping limbs 40 and the leaf spring elements 46 in each case is a fixed one, i.e. not an articulated one. The leaf spring elements 46 are curved such that they extend in the distal direction and thereby distance themselves from the inner surfaces, i.e. the surfaces of the gripping limbs 40 which face one another, so that the two leaf spring elements 46 meet in an articulation point 48, at which they are connected in an articulated manner to the proximal end of the push rod 20. Thereby, the two leaf spring elements 46 are connected to the push rod 20 at a common articulation point 48. The articulation point 48 lies on the instrument longitudinal axis X and is distanced from the articulation points 42 in the proximal direction and in the direction of this axis.

For opening the jaw 10, the two gripping limbs 40 are pressed together, i.e. are moved to one another, so that with the respective pivoting movement about the articulation points 42, the proximal ends of the gripping limbs 40 approach one another. The two leaf spring elements 46 are deformed with this pivoting movement, wherein due to the fixed connection at the proximal end, the distal ends of the leaf spring elements 46 with the articulation point 48 displace distally, by which means the push rod 20 is advanced in the distal direction and the actuation element 22 pushes against the actuation surfaces 28 of the jaw parts 6 and 8. Thereby, the jaw parts 6 and 8 are then opened by way of elastic deformation. For the closure of the jaw 10, the gripping limbs 40 are let go of, so that on account of the spring effect of the leaf spring elements 46, they move back into their initial position, i.e. pivot apart. Thereby, the push rod 20 is then pulled back in the proximal direction. Due to the elastic bias of the jaw parts 6 and 8 which is described above, these are then held in bearing contact in a secure manner without further blocking elements, so that for example a needle 38 remains securely clamped between the gripping sections 12 and 14. For release, the gripping limbs 40 merely need to be pressed, without further blocking or release elements having to be actuated.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

I claim:

1. A medical gripper instrument with two jaw parts (6, 8) comprising distal gripping sections (12, 14) lying opposite one another, wherein at least one of the jaw parts (6, 8) comprises a proximal holding section (16, 18) which is elastically deformable in a manner such that the distal gripping sections (12, 14) of the two jaw parts (6, 8) are capable of being moved to one another and away from one another by way of elastic deformation of the at least one holding section (16, 18).

2. The medical gripper instrument according to claim 1, wherein both jaw parts (6, 8) include the proximal holding section (16, 18) which are elastically deformable in a manner such that the gripping sections (12, 14) of the two jaw parts (6, 8) are capable of being moved to one another and away from one another by way of elastic deformation of both holding sections (16, 18).

3. The medical gripper instrument according to claim 1, wherein the gripping sections (12, 14) of the two jaw parts (6, 8) bear on one another in an idle position and are capable of being moved apart by way of elastic deformation of the at least one holding section (16, 18).

4. The medical gripper instrument according to claim 2, wherein in an idle position, the at least one holding section (16, 18) of a jaw part (6, 8) is elastically deformed in a manner such that the at least one holding section (16, 18) produces a spring force which holds the gripping sections (12, 14) of the two jaw parts (6, 8) in bearing contact.

5. The medical gripper instrument according to claim 1, further comprising an actuation element (22), being movable linearly in the direction of a tip thereof, between the jaw parts (6, 8), in a manner such that the gripping sections (12, 14) of the two jaw parts (6, 8) are moved apart amid elastic bending of at least one holding section (16, 18).

6. The medical gripper instrument according to claim 1, further comprising an actuation rod (20), which is movable in the direction of a longitudinal axis X of the instrument and which at its proximal end is connected to a handle (4), for moving the gripping section (12, 14) at least of one of the jaw parts (6, 8).

7. The medical gripper instrument according to claim 1, wherein by a handle (4) which comprises at least one gripping limb (40) which is mounted in an articulated manner at a distal end thereof and is connected at a proximal end thereof to a leaf spring element (46) which extends distally proceeding from the proximal end of the gripping limb (40) and with the distal end distanced to the gripping limb (40), is connected to the proximal end of an actuation rod (20).

8. The medical gripper instrument according to claim 7, wherein the connection of the leaf spring element (46) to the proximal end of the actuation rod (20) is distanced, in the proximal direction, to the articulated mounting (42) of the gripping limb (40).

9. The medical gripper instrument according to claim 1, wherein the instrument is a needle take-over instrument.
